(19) United States
(12) Patent Application Publication
(10) Pub. No.: US 2022/0095992 A1
(43) Pub. Date: Mar. 31, 2022

(54) BRAIN ACTIVITY MONITORING

(71) Applicant: Commonwealth Scientific and Industrial Research Organisation, Acton, Australian Capital Territory (AU)

(72) Inventors: Umut GUVENC, Acton, Australian Capital Territory (AU); Paulo DESOUZA, Acton, Australian Capital Territory (AU); Ayobami IJI, Acton, Australian Capital Territory (AU)

(73) Assignee: Commonwealth Scientific and Industrial Research Organisation, Acton, Australian Capital Territory (AU)

(21) Appl. No.: 17/425,074

(22) PCT Filed: Feb. 5, 2020
(86) PCT No.: PCT/AU2020/050088

§ 371 Date: Jul. 22, 2021

(30) Foreign Application Priority Data
Feb. 5, 2019 (AU) 2019900357

(51) Int. Cl. A61B 5/00; A61B 5/145; A61B 5/293

(52) U.S. Cl. CPC A61B 5/4094; A61B 2560/0209; A61B 5/14539; A61B 5/7221; A61B 5/293; A61B 5/7264; A61B 5/0006; A61B 5/002; A61B 5/0022; A61B 5/742; A61B 5/7225; A61B 5/747; A61B 2562/0247; A61B 2562/0271; A61B 2562/046; A61B 2560/0219; A61B 5/6868

(57) ABSTRACT

A system for monitoring brain activity of a subject, including an implantable measurement device including: a sensor configured to measure electrical activity in the brain; electronic measurement processing devices configured to: receive measurement data from the sensor; use the measurement data to determine if the brain activity of the subject is indicative of an event; and generate event data indicative of the brain activity associated with the event; and an implantable transceiver configured to transmit the event data; an inductive implantable coil configured to inductively receive power; and an external monitoring device including: an external transceiver configured to receive event data from the implantable measurement device; an inductive external coil configured to inductively transmit power to the implantable measurement device; and electronic monitoring processing devices configured to: generate subject data; and transfer the subject data to analysis processing devices for analysis.

### BACKGROUND OF THE INVENTION

The present invention relates to a system and method for monitoring brain activity, and in one particular example, a system and method including an implantable measuring device for monitoring brain activity indicative of an event.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

Traumatic brain injury (TBI) is an unfortunate incident that often requires more than one surgery. It is apparent that the surgery is operated on a delicate area, and the operations are generally spread out a longer period of time so that the brain can recover before the next operation. Therefore, the patients often spend an unpleasant couple of years until the full recovery. In the specific case of TBI, the patients undergo two surgeries. One is called decompressive craniectomy, in which a part of the skull is removed to release the increasing internal pressure due to swelling of the brain immediately after the incident. The second is called cranioplasty in which a prosthesis is placed instead of the removed skull after the swelling of the brain eases and the brain returns to its normal size.

Generally, patients undergo the first surgery immediately after the incident. In the surgery, a part of the skull is removed to allow the brain to swell. This minimise any further damages caused by the swelling of the brain creating internal high pressure. A protective helmet, such as DuraShield™, is placed to preserve distinction of the skin and the dura matter post surgery. Patients spend a long time, up to a year, waiting for the swelling of their brain to ease while having a part of their skull removed and wearing the protective helmet instead. Once the swelling has eased, another surgery can be performed to place a permanent protective prosthesis instead of the removed skull.

The recovery period after the first surgery is critical in terms of monitoring the health of the brain. During this time, about one third of the patients develop post-traumatic epilepsy. Thus, it is crucial to continuously and precisely monitor the brain activity in order to apply proper treatment at the appropriate time to prevent the onset of epilepsy. Currently, patients' brain activities are monitored with electroencephalogram (EEG) monitoring systems which require wired connections between the patient and bulky hospital equipment. As such, the current general practice is not mobile and does not provide continuous monitoring capability.

### SUMMARY OF THE PRESENT INVENTION

In one broad form an aspect of the present invention seeks to provide a system for monitoring brain activity of a subject, the system including: at least one implantable measurement device including: a sensor configured to measure electrical activity in the brain; one or more electronic measurement processing devices configured to: receive measurement data from the sensor; use the measurement data to determine if the brain activity of the subject is indicative of an event; and generate event data indicative of the brain activity associated with the event; and an implantable transceiver configured to transmit the event data; an inductive implantable coil configured to inductively receive power; and, an external monitoring device including: an external transceiver configured to receive event data from the implantable measurement device; an inductive external coil configured to inductively transmit power to the implantable measurement device; and, one or more electronic monitoring processing devices configured to: generate subject data; and, transfer the subject data to one or more analysis processing devices for analysis.

In one embodiment the at least one implantable measurement device is placed under a skull of the subject.

In one embodiment the at least one implantable measurement device is placed on dura mater of the subject.

In one embodiment the at least one implantable measurement device is at least partially embedded in a protector shield protecting the brain.

In one embodiment the implantable measurement device includes at least one of: a temperature sensor, the measurement data being indicative of a temperature; a pressure sensor, the measurement data being indicative of a pressure; and, a pH level sensor, the measurement data being indicative of a pH level.

In one embodiment the sensor includes one or more electrodes.

In one embodiment the sensor includes four electrodes.

In one embodiment the one or more electronic measurement processing devices have a physical footprint of approximately 4 mm×4 mm.

In one embodiment the sensor is mounted on the one or more electronic measurement processing devices.

In one embodiment the at least one implantable measurement device includes one or more amplifiers, each amplifier being configured to amplify measurement signals from a respective electrode.

In one embodiment the at least one implantable measurement device includes an array selection module for selectively activating the one or more amplifiers.

In one embodiment the at least one implantable measurement device includes a sampling module configured to sample a measurement signal from the sensor.

In one embodiment the sampling module includes an analog-to-digital converter.

In one embodiment the sampling module includes a coarse analog-to-digital converter and a fine analog-to-digital converter.

In one embodiment the at least one implantable measurement device includes a temporary memory for storing the measurement data.

In one embodiment the event includes a seizure.

In one embodiment the at least one implantable measurement device includes a data quality module for identifying inaccurate sensing from an electrode.

In one embodiment, the data quality module is configured to perform at least a first data quality test.

In one embodiment, the first data quality test includes determining mean and standard deviation values from peak amplitude values in EEG data measured from the electrode.

In one embodiment, the first data quality test further includes determining whether the determined mean and standard deviation values are within a predetermined range with a fuzzy-logic system.

In one embodiment, the data quality module is configured to perform at least a second data quality test.

In one embodiment, the second data quality includes determining a distance between consecutive EEG signal peaks measured via the electrode and calculating an average EEG wave.

In one embodiment, the second data quality test further includes comparing the average EEG wave of the electrode against a control EEG wave.

In one embodiment, the control EEG wave is preloaded onto a memory of the implantable measurement device.

In one embodiment, the control EEG wave includes an average EEG wave calculated based on measurements of one or more further electrodes.

In one embodiment the at least one implantable measurement device includes a data memory for storing the event data.

In one embodiment at least one implantable measurement device includes an encryption module for encrypting the event data before transmitting.

In one embodiment the implantable transceiver includes an implantable antenna.

In one embodiment the at least one implantable measurement device includes an implantable energy storage unit.

In one embodiment the implantable energy storage unit is a micro-battery.

In one embodiment the implantable energy storage unit is a three-dimensional micro-battery.

In one embodiment the at least one implantable measurement device has a physical footprint of approximately 14 mm×7.2 mm.

In one embodiment the external transceiver includes an external antenna.

In one embodiment the external monitoring device includes an external power amplifier inductively connected to the inductive external coil.

In one embodiment the external monitoring device includes an external energy storage unit providing energy to the inductive external coil.

In one embodiment the external energy storage unit is a micro-battery.

In one embodiment the external energy storage unit is a three-dimensional micro-battery.

In one embodiment the external monitoring device is placed in a headgear wearable by the subject.

In one embodiment the one or more electronic measurement processing devices configured to be operable in a standby mode and an active mode depending on whether an event is occurring.

In one embodiment, when in the standby mode, the one or more electronic measurement processing devices are configured to: identify inaccurate sensing from an electrode; select one or more amplifiers based on identified inaccurate sensing; receive measurement signals from the selected amplifiers; filter the measurement signals; sample the measurement signals at a low sampling rate and/or a coarse sampling resolution; and store sampled signal data in a temporary memory.

In one embodiment the one or more electronic measurement processing devices are configured to: at least partially analyse the sampled signal data; determine if an event is occurring in accordance with results of the analysis; and, if an event is occurring, switch to the active mode.

In one embodiment the one or more electronic measurement processing devices are configured to determine if an event is occurring by at least one of: analysing one or more parameters derived from the sampled signal data; comparing the sampled signal data to previous sampled signal data; and, using machine learning techniques.

In one embodiment the low sampling rate is approximately 512 Hz, and the coarse sampling resolution is 8-bit.

In one embodiment, when in the active mode, the one or more electronic measurement processing devices are configured to: select all available amplifiers; receive measurement signal from the selected amplifiers; filter the measurement signal; sample the measurement signal at a high sampling rate and/or a fine sampling resolution; and store event data including sampled signal data in a data memory.

In one embodiment the high sampling rate is approximately 10 kHz, and the fine sampling resolution is 16-bit.

In one embodiment the one or more electronic measurement processing devices are configured to encrypt event data in the data memory.

In one embodiment the implantable measurement device is configured to be operable in a receiving mode and a transmitting mode according to a transmission request.

In one embodiment, when in the receiving mode, the implantable transceiver is configured to receive the transmission request from the external monitoring device.

In one embodiment, when in the transmitting mode, the implantable transceiver is configured to transmit the event data to the external monitoring device.

In one embodiment the system further includes a plurality of implantable measurement devices.

In one embodiment the system includes one or more processing systems configured to: at least partially analyse the subject data; and generate activity data indicative of results of the analysis.

In one embodiment the one or more processing systems at least partially analyse the subject data using machine learning.

In one embodiment the system further includes a client device configured to interface with a user.

In one embodiment the client device includes: a graphical user interface; a client device transceiver for receiving the subject data from the external monitoring device; and a client device display for displaying an activity indicator indicative of brain activity based on the subject data.

In one embodiment the client device is one of a tablet, a smartphone, a smart watch and a computer.

In one embodiment the client device is configured to: transmit subject data to one or more processing systems; receive activity data from the one or more processing systems; and, display an activity indicator based on the activity data.

In one broad form an aspect of the present invention seeks to provide an implantable measurement device, including: a sensor configured to measure electrical activity in the brain; one or more electronic measurement processing devices configured to: receive measurement data from the sensor; use the measurement data to determine if the brain activity of the subject is indicative of an event; and generate event data indicative of the brain activity associated with the event; and an implantable transceiver configured to transmit the event data; an inductive implantable coil configured to inductively receive power.

In one embodiment the implantable measurement device is placed under a skull of the subject.

In one embodiment the implantable measurement device is placed on dura mater of the subject.

In one embodiment the implantable measurement device is at least partially embedded in a protector shield protecting the brain.

In one embodiment the implantable measurement device includes at least one of: a temperature sensor, the measurement data being indicative of a temperature; a pressure sensor, the measurement data being indicative of a pressure; and, a pH level sensor, the measurement data being indicative of a pH level.

In one embodiment the sensor includes one or more electrodes.

In one embodiment the sensor includes four electrodes.

In one embodiment the one or more electronic measurement processing devices have a physical footprint of approximately 4 mm×4 mm.

In one embodiment the sensor is mounted on the one or more electronic measurement processing devices.

In one embodiment the implantable measurement device includes one or more amplifiers, each amplifier being configured to amplify measurement signals from a respective electrode.

In one embodiment the implantable measurement device includes an array selection module for selectively activating the one or more amplifiers.

In one embodiment the implantable measurement device includes a sampling module configured to sample a measurement signal from the sensor.

In one embodiment the sampling module includes an analog-to-digital converter.

In one embodiment the sampling module includes a coarse analog-to-digital converter and a fine analog-to-digital converter.

In one embodiment the implantable measurement device includes a temporary memory for storing the measurement data.

In one embodiment the event includes a seizure.

In one embodiment the implantable measurement device includes a data quality module for identifying inaccurate sensing from an electrode.

In one embodiment the implantable measurement device includes a data memory for storing the event data.

In one embodiment implantable measurement device includes an encryption module for encrypting the event data before transmitting.

In one embodiment the implantable transceiver includes an implantable antenna.

In one embodiment the implantable measurement device includes an implantable energy storage unit.

In one embodiment the implantable energy storage unit is a three-dimensional micro-battery.

In one embodiment the implantable measurement device has a physical footprint of approximately 14 mm×7.2 mm.

In one embodiment the one or more electronic measurement processing devices configured to be operable in a standby mode and an active mode depending on whether an event is occurring.

In one embodiment, when in the standby mode, the one or more electronic measurement processing devices are configured to: identify inaccurate sensing from an electrode; select one or more amplifiers based on identified inaccurate sensing; receive measurement signals from the selected amplifiers; filter the measurement signals; sample the measurement signals at a low sampling rate and/or a coarse sampling resolution; and store sampled signal data in a temporary memory.

In one embodiment the one or more electronic measurement processing devices are configured to: at least partially analyse the sampled signal data; determine if an event is occurring in accordance with results of the analysis; and, if an event is occurring, switch to the active mode.

In one embodiment the one or more electronic measurement processing devices are configured to determine if an event is occurring by at least one of: analysing one or more parameters derived from the sampled signal data; comparing the sampled signal data to previous sampled signal data; and, using machine learning techniques.

In one embodiment the low sampling rate is approximately 512 Hz, and the coarse sampling resolution is 8-bit.

In one embodiment, when in the active mode, the one or more electronic measurement processing devices are configured to: select all available amplifiers; receive measurement signal from the selected amplifiers; filter the measurement signal; sample the measurement signal at a high sampling rate and/or a fine sampling resolution; and store event data including sampled signal data in a data memory.

In one embodiment the high sampling rate is approximately 10 kHz, and the fine sampling resolution is 16-bit.

In one embodiment the one or more electronic measurement processing devices are configured to encrypt event data in the data memory.

In one embodiment the implantable measurement device is configured to be operable in a receiving mode and a transmitting mode according to a transmission request.

In one embodiment, when in the receiving mode, the implantable transceiver is configured to receive the transmission request from an external monitoring device.

In one embodiment, when in the transmitting mode, the implantable transceiver is configured to transmit the event data to an external monitoring device.

In one broad form an aspect of the present invention seeks to provide an external monitoring device, including: an external transceiver configured to receive event data from the implantable measurement device; an inductive external coil configured to inductively transmit power to the implantable measurement device; and, one or more electronic monitoring processing devices configured to: generate subject data; and, transfer the subject data to one or more analysis processing devices for analysis.

In one embodiment the external transceiver includes an external antenna.

In one embodiment the external monitoring device includes an external power amplifier inductively connected to the inductive external coil.

In one embodiment the external monitoring device includes an external energy storage unit providing energy to the inductive external coil.

In one embodiment the external energy storage unit is a micro-battery.

In one embodiment the external energy storage unit is a three-dimensional micro-battery.

In one embodiment the external monitoring device is placed in a headgear wearable by the subject.

In one embodiment the external monitoring device is configured to transmit a transmission request to an implantable measurement device.

In one broad form an aspect of the present invention seeks to provide a method of monitoring brain activity of a subject, the method including: in at least one implantable measurement device including: a sensor configured to be placed in proximity of the brain of the subject to measure electrical activity in the brain; an implantable transceiver; an inductive implantable coil; and, one or more electronic measurement processing devices, the method including: receiving measurement data from the sensor; using the measurement data to determine if the brain activity of the subject is indicative of an event; and generating event data indicative of the brain activity associated with the event; and transmitting the event data; and, in an external monitoring device including: an external transceiver configured to receive event data from the implantable measurement device; an inductive external coil configured to inductively transmit power to the implantable measurement device; and, one or more electronic monitoring processing devices, the method including: generating subject data; and, transferring the subject data to one or more analysis processing devices for analysis.

It will be appreciated that the broad forms of the invention and their respective features can be used in conjunction, interchangeably and/or independently, and reference to separate broad forms is not intended to be limiting.

### BRIEF DESCRIPTION OF THE DRAWINGS

Various examples and embodiments of the present invention will now be described with reference to the accompanying drawings, in which: —

FIG. 1 is a schematic diagram of an example of a system for monitoring brain activity of a subject;

FIG. 2 is a flow chart of an example of a method for monitoring brain activity of a subject;

FIG. 3 is a schematic diagram of an example of a network architecture;

FIG. 4 is a schematic diagram of an example of a processing system;

FIG. 5 is a schematic diagram of an example of a client device;

FIGS. 6A and 6B are schematic diagrams of an example of a system for monitoring brain activity of a subject;

FIGS. 7A to 7C are schematic diagrams of an example of internal components in an implantable monitoring device;

FIG. 8 is a flow chart of an example of a process for operating a brain monitoring system;

FIGS. 9A and 9B are a flow chart of a specific example of a process for monitoring brain activity;

FIG. 10 is a flow chart of an example of a process for operating a brain activity analysing system;

FIG. 11 is a block diagram for service based architecture;

FIG. 12A is an example of EEG data;

FIG. 12B is a histogram displaying the distribution of peak intensities of the EEG data in FIG. 12A;

FIG. 12C is the peak by peak measurement of standard deviations;

FIG. 13 is a block diagram of a system model for seizure detection and prediction;

FIG. 14 is a diagram illustrating seizure detection function; and,

FIG. 15 is a sequence of outputs from the seizure detection module.

### DETAILED DESCRIPTION OF THE PREFERRED EMBODIMENTS

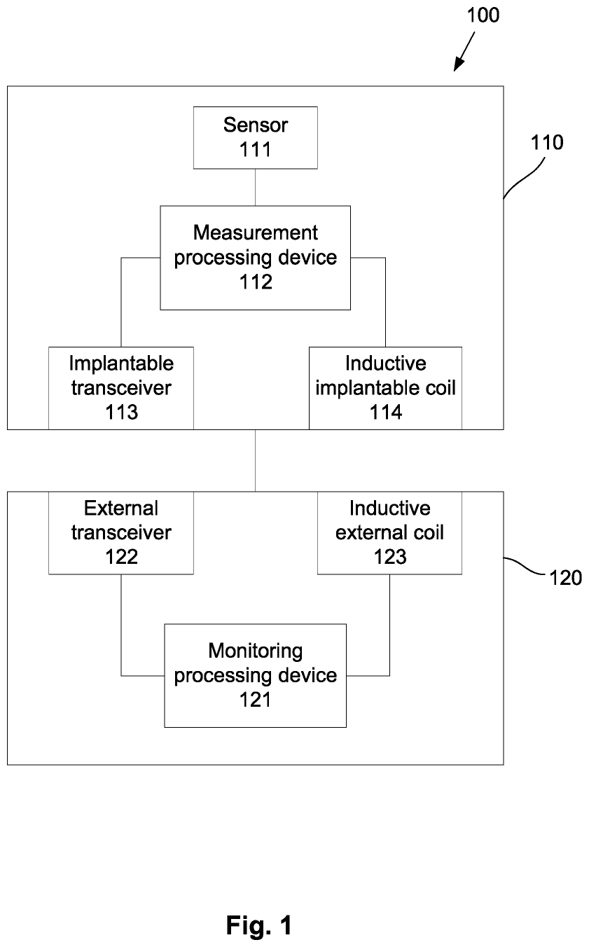

An example of a system for monitoring brain activity of a subject will now be described with reference to FIG. 1.

The system 100 includes an implantable measuring device 110, which is configured to be implanted on the subject being monitored, and an external monitoring device 120 in communication with the implantable measuring device 110.

The implantable measurement device 110 includes a sensor 111, one or more electronic measurement processing devices 112, an implantable transceiver 113 and an inductive implantable coil 114. The external monitoring device 120 includes an external transceiver 122, an inductive external coil 123, and one or more electronic monitoring processing devices 121.

The sensor 111 typically placed in proximity with the brain of the subject, the sensor being configured to measure electrical activity in the brain. The sensor 111 may be an electrode or a number of electrodes, or any other suitable sensor that is implantable and capable of providing measurements of electrical activity of the brain, could be used. The sensor 111 is configured to provide measurements with suitable temporal and spatial resolutions. In one example, the sensor 111 is a biocompatible electrode with a temporal resolution of approximately 10 kHz and a spatial resolution such as 1 cm, which would typically be tailored depending on requirements for the subject, but it will be appreciated that other arrangements could be used. It will also be appreciated that other sensors could be provided for measuring other indicators of activity such as indications of temperature, pressure, pH level, or the like.

The one or more electronic measurement processing devices 112 are configured to receive measurement data from the sensor 111, use the measurement data to determine if the brain activity of the subject is indicative of an event and generate event data indicative of the brain activity associated with the event. Accordingly, the one or more measurement processing device 112 may be formed from any suitable processing device that is capable of processing measurement data, and could include a microprocessor, microchip processor, logic gate configuration, firmware optionally associated with implementing logic such as an FPGA (Field Programmable Gate Array), or any other electronic device, system or arrangement. Furthermore, for ease of illustration the remaining description will refer to a processing device, but it will be appreciated that multiple processing devices could be used, with processing distributed between the devices as needed, and that reference to the singular encompasses the plural arrangement and vice versa.

The implantable transceiver 113 transmits the event data to the external transceiver 122, and it will be appreciated that the transceivers could be any form of transceiver, but typically are short range low power wireless transceiver. The implantable transceiver 113 and the external transceiver 122 may be formed of an integral part of the measurement processing device 112 and the monitoring processing device 121, respectively, or may be separate components.

The one or more electronic monitoring processing devices 121 generate subject data using received event data and transfer the subject data to one or more analysis processing devices for analysis. Accordingly, the one or more measurement processing device 112 may be formed from any suitable processing device that is capable of processing measurement data, and could include a microprocessor, microchip processor, logic gate configuration, firmware optionally associated with implementing logic such as an FPGA (Field Programmable Gate Array), or any other electronic device, system or arrangement.

The inductive implantable coil 114 is configured to inductively receive power from the inductive external coil 123 allowing energy to be provided to the implantable measurement device 110. The coils may be coils capable of operating at a suitable carrier frequency and delivering suitable power. In one example, the inductive coil operates in 13.56 MHz and delivers up to 25 mW of power with an efficiency of approximately 40% within a distance of 1 cm.

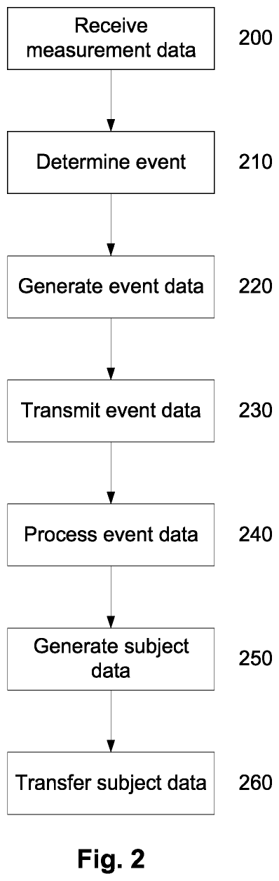

An example of operation of the system 100 will now be described with reference to FIG. 2.

In this example, at step 200, the electronic measurement processing device 112 receives measurement data, such as raw electrocorticographical (ECoG) or intracranial electroencephalographical (iEEG) signal, from the sensor 111. In one example, the electronic measurement processing device 112 receives the ECoG signal from an electrode placed in proximity with, and optionally in contact with, the brain.

At step 210, the electronic measurement processing device 112 uses the measurement data to determine if the brain activity of the subject is indicative of an event, such as a seizure, onset of a seizure, likelihood of a seizure, or the like. In one example, such events are characterised by a particular pattern and/or change in brain activity, allowing this to be identified by the electronic measurement processing device 112. If an event is not occurring the electronic measurement processing device 112 can return to step 200 to allow this process to be repeated.

At step 220, assuming an event is occurring, the electronic measurement processing device 112 generates event data indicative of the brain activity associated with the event. In one example, the event data could be the measured electrical signals, sensor information, and/or other parameters derived therefrom, such as signal frequency and/or signal power parameters derived from the measured electrical signals.

At step 230, the event data is transmitted from the implantable measurement device 110 to the external monitoring device 120, via the implantable transceiver 113 and the external transceiver 122.

Upon receiving the event data, at step 240, the electronic monitoring processing device 121 processes the event data, and generates subject data at step 250. In one example, the subject data could simply be the event data, in which case minimal processing is performed, such as decrypt and/or validate all data is received. More typically however, the subject data includes subject information, such as a subject identifier, as well as the event data and/or other parameters derived therefrom.

At step 260, the subject data is then transferred for further processing, analysing and/or displaying, which could be achieved in any suitable manner, such as by using another external transceiver, or the like. For example, this could include transmitting the subject data to a client device, such as a mobile phone, tablet or computer, allowing an activity indicator indicative of brain activity to be displayed to a user, such as a medical practitioner and/or the subject. This process can also include transmitting the subject data to one or more processing systems, such as servers, for more detailed analysis.

Accordingly, the system 100 is capable of measuring electrical activity, such as ECoG signals, using sensors 111 placed in proximity of the brain, which can therefore allow measurements to be performed with a higher spatial resolution when comparing to conventional electroencephalography (EEG) where the sensors are placed on skin/scalp. Such measurements can provide an imaging advantage for pre-surgical planning or for monitoring post-surgical recovery, as they provide a more accurate representation of activity within the brain, and are more sensitive to changed conditions, allowing events, such as seizures, or the like, to be detected more accurately and rapidly.

Furthermore, the implantable measurement device 110 includes an electronic measurement processing device 112 which is capable of on-chip processing. This allows the implantable measurement device 110 to at least partially process the measurement data before transmitting to an external processor. As such, the amount of data for transmitting is reduced and therefore minimising power consumption. Since minimal power is required, the power can be inductively supplied with power from the external monitoring device, which allows the weight and size of the implantable measurement device to be reduced. This also allows multiple implantable measurement devices 110 to be powered by a single external monitoring device 120, which can in turn help improve the resolution of measurements that can be performed, allowing the detection of events to be performed more accurately.

This arrangement can be used in a post-surgical scenario, where part of the skull has been removed, allowing the brain to be monitored to detect seizure events, and potentially the onset of seizures more effectively than is currently the case.

A number of further features will now be described.

In one example, the implantable include measurement device includes a temperature sensor, a pressure sensor or a pH level sensor, in which case the measurement data is also indicative of a temperature, a pressure or a pH level. In this case, these parameters could be analysed in conjunction with the electrical activity to further refine the ability to detect events.

The implantable measurement device may be placed under skull of the subject, below dura mater of the brain, directly on dura mater, or embedded in a protector shield such as DuraShield™ by Anatomies® Pty Ltd. This allows the sensor to more accurately measure the electrical activity of the brain by being proximal to the brain. Additionally, the implantable measurement device may be placed post-surgery when placing the protector shield, thereby simplifying the implanting process.

In one example, the sensor may include one or more electrodes to provide one or more channels providing measurement data to the electronic monitoring processing device. In one example, the sensor includes four electrodes to provide the measurement data with four channels. The use of multiple channels in this manner improves measurement resolution and allows for redundancy, for example, allowing channels to be ignored if measurements are inaccurate.

The electronic measurement processing device may have a physical footprint of approximately 4 mm×4 mm. This allows a plurality of electronic measurement processing devices to be implanted and therefore increase the spatial resolution.

In one example, the electronic measurement processing device is placed on the sensor, so that the footprint of the implantable measurement device is minimised.

The implantable measurement device, and in one example, the electronic measurement processing device may include one or more amplifiers, each amplifier being configured to amplify measurement signals from a respective electrode. This allows the raw electrical signal to be individually amplified, avoiding interference between channels, and avoiding the need for switching to be used to sample different channels.

Furthermore, the implantable measurement device, and in one example, the electronic measurement processing device may include an array selection module for selectively activating the amplifiers. This allows the electronic measurement processing device to selectively receive the measurement signal by selecting the amplifiers, so that channels can be ignored in the event that measurement signals are deemed to be inaccurate.

The implantable measurement device, and in one example, the electronic measurement processing device may further include a sampling module to sample a measurement signal. In one example, the sampling module includes an analog-to-digital converter, which allows the electronic measurement processing device to convert the measurement signal to digital signal for further processing. In one example, the sampling module includes a coarse analog-to-digital converter and a fine analog-to-digital converter. This allows the electronic measurement processing device to selectively sampling the measurement signal with different sampling frequencies. For example, coarse sampling can be used when assessing if an event is occurring, whereas fine sampling may be used to record measurement signals during the event, allowing electrical activity during events to be captured in greater detail, which can in turn allow more in depth clinical assessment of the event to be performed.

The implantable measurement device, and in one example, the electronic measurement processing device may further include a temporary memory for storing the measurement data. The temporary memory may be able to store approximately 1 minute of the measurement data, which allows a limited amount of measurement data to be recovered. This can also be used to allow changes in measurement data over time to be analysed, which can in turn assist with identifying events.

The implantable measurement device, and in one example, the electronic measurement processing device, may further include a data quality module for identifying inaccurate sensing from an electrode, which may arise for a variety of reasons, such as an inaccurate amplifier, poor electrode contact with the brain, or the like, and which allows the electronic measurement processing device to only receive the measurement signal from accurate channels.

In one example, the implantable measurement device may include a data memory for storing the event data. This allows the event data to be stored before transmitting, for example to allow transmission to occur at a suitable time, such as when data is not being recorded, thereby allowing power requirements to be minimised. This also allows an encryption module to be used to encrypt the event data before its transmission. As the event data may include sensitive information relating to the subject, this allows the event data to be transmitted securely.

In one example, the implantable transceiver may include an implantable antenna for wireless transmission with the external transceiver including a similar corresponding external antenna.

In one example, the implantable measurement device has a physical footprint of approximately 14 mm×7.2 mm. This allows a plurality of implantable measurement device to be implanted and therefore increase the spatial coverage.

In one example, the external monitoring device may include an external power amplifier inductively connected to the inductive external coil, so that sufficient power can be supplied to a receiver coil in the implantable measurement device. Furthermore, the external monitoring device may include an energy storage unit, such as a battery, lithium-ion batteries or any other suitable energy storage units, so that sufficient energy can be provided to the external monitoring device and the receiver coil in the implantable measurement device. It will be appreciated that an energy storage unit such as a micro-battery or a 3-D micro-battery may be provided in the implantable measurement device.

In one example, the external monitoring device is placed in a headgear wearable by the subject. This allows the external monitoring device to be in proximity to the implantable measurement device, so that power can be inductively and efficiently transferred. Such headgear is often worn following surgical procedures, and this therefore allows the system to be easily integrated into existing headgear, so that there is no additional burden in using the system.

In one example, the electronic measurement processing device may be operable in a standby mode and an active mode depending on whether an event is occurring. Having the two modes allows the electronic measurement processing device to manage power consumption, thereby reduce overall power usage, and allowing inductive powering of the measuring device.

When in the standby mode, the electronic measurement processing device identifies inaccurate sensing from an electrode, selects the amplifiers based on any detected inaccurate sensing, receives measurement signal from the selected amplifiers, filters the measurement signal, samples the measurement signal at a low sampling rate and/or a coarse sampling resolution, and store sampled signal data in a temporary memory. This allows the electronic measurement processing device to monitor the brain activity with minimal power consumption, by selecting a portion of the amplifiers and sampling at the low sampling rate and/or the coarse sampling resolution, such as approximately 512 Hz with 8-bit resolution.

In one example, the electronic measurement processing device at least partially analyses the sampled signal data, determines if an event is occurring in accordance with results of the analysis and if an event is occurring, and subsequently switches to the active mode. In particular, the electronic measurement processing device determines if the event is occurring using the sampled signal data. This can be achieved using a variety of techniques, such as analysing one or more parameters derived from the sampled signal data, comparing the sampled signal data to previous data and/or using machine learning techniques. If it is determined that an event is occurring, the active mode can be triggered to allow additional data to be collected.

Specifically, when in the active mode, the electronic measurement processing device selects all available amplifiers, receives measurement signals from the selected amplifiers, filters the measurement signals, samples the measurement signal at a high sampling rate and/or a fine sampling resolution, and stores sampled signal in a data memory. This allows the electronic measurement processing device to capture a more complete measurement data sample or set when the event is occurring, by selecting all available amplifiers and sampling at the high sampling rate and/or the fine resolution, such as approximately 10 kHz with 16-bit resolution.

In one example, the electronic measurement processing device encrypts sampled data in the data memory. This can be achieved using a session key exchanged with the external monitoring device, so that a unique encryption key is used each time data is being transferred. In this instance, the session key is typically generated by the implantable measurement device and encrypted using a public key of the external monitoring device, so that only the external monitoring device can decrypt the session key. Such encryption mechanisms avoid third parties intercepting and/or interpreting sensitive patient data. It will be appreciated that a two-way authentication encryption or other suitable encryption protocols may also be used.

In one example, the implantable measurement device may be operable in a receiving mode and a transmitting mode according to a transmission request. When in the receiving mode, the implantable transceiver receives the transmission request from the external monitoring device. Upon receiving the transmission request, the implantable measurement device switches to the transmitting mode. When in the transmitting mode, the implantable transceiver transmits the event data to the external monitoring device. Accordingly, the external monitoring device transmits a transmission request to the implantable measurement device. In one example, the transmission request is generated by the external monitoring device, although alternatively, the transmission request is received from another device and then transmitted to the implantable measurement device by the external monitoring device. This allows power consumed for transmission by the implantable measurement device to be controlled externally by the external monitoring device, and therefore facilitates efficient power management.

In one example, the system includes a plurality of implantable measurement devices. This allows the system to provide normal function when one or more of the implantable measurement devices fail or when the quality of the measurements become unacceptable in time.

As previously mentioned, the subject data can be transmitted for further analysis. In one example, this is achieved using one or more processing systems configured to at least partially analyse the subject data and generate activity data indicative of results of the analysis.

The analysis can be of any performed in any appropriate manner, depending on the preferred implementation. In one example, the subject data is analysed to determine one or more metrics, which could be parameters relating to attributes of the measurement signals, such as frequency components, power factor analysis, or the like. The metrics can then be compared to reference metrics, such as previous parameter values and/or parameter values derived from sample populations, in order to help characterise the event, for example in order to determine an event status, including a type and/or severity of an event.

In one example, this is performed at least in part using a computational model embodying a relationship between the metrics and an event status. The computational model can be derived by analysing subject data from multiple subjects. In one example, this is performed using machine learning, for example, by training a reference model using subject data from one or more different subjects. The nature of the model and the training performed can be of any appropriate form and could include any one or more of decision tree learning, random forest, logistic regression, association rule learning, artificial neural networks, deep learning, inductive logic programming, support vector machines, clustering, Bayesian networks, reinforcement learning, representation learning, similarity and metric learning, genetic algorithms, rule-based machine learning, learning classifier systems, or the like. As such schemes are known, these will not be described in any further detail. In one example, this can include training a single model to determine the medication state using metrics from reference subjects with a combination of healthy and unhealthy medication states, although this is not essential and other approaches could be used. By using machine learning, this can improve the accuracy of analysis and also expand the complexity of the analysis.

Activity data could be displayed locally, or could be transferred to other devices allowing the activity data to be displayed, and/or allowing alerts or other notifications to be provided to a clinician and/or the subject.

In one example, the system may include a client device to interface with a user, such as a physician or the subject. The client device may include a graphical user interface, a client device transceiver for receiving the subject data from the external monitoring device, and a client device display for displaying an activity indicator based on the subject data. In one example, the client device may be a tablet, a smartphone, a smart watch or a computer. This allows the user to at least view activity indicators, such as details of events, including an event type, severity, duration, or the like, based on or derived from the subject data.

In one example, the client device may upload client data to the one or more processing systems, such as a server. This allows further processing to be carried out at the server which may have higher processing power. Accordingly, the server may include one or more analysis processing devices to at least partially analyse the client data and generate the activity data. The client device can then receive the activity data and display an activity indicator, including a report and/or an indication of an event status. Thus, the client server may transmit the reporting data to a client device for displaying result data, so that a user, such as a physician or the subject, may access the reporting data.

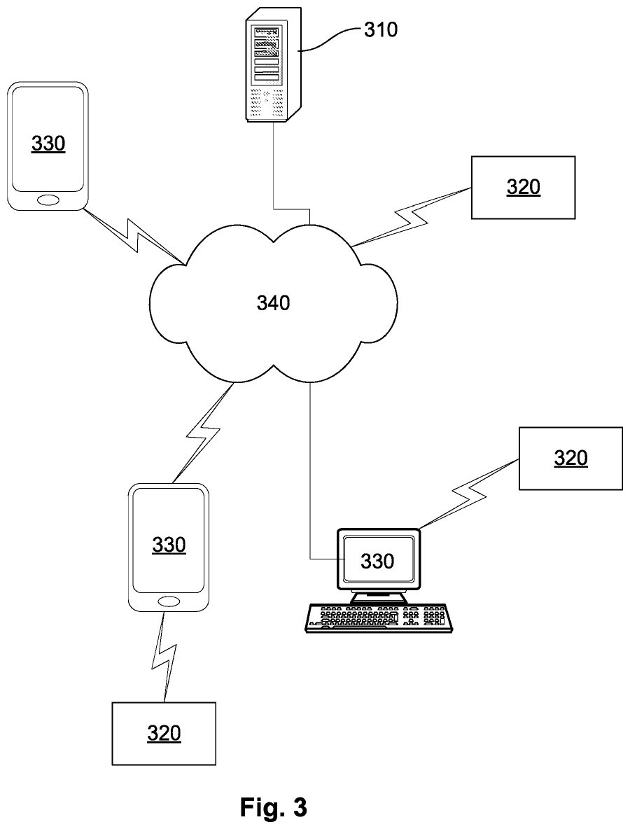

An example of an analysing system will now be described in more detail with reference to FIG. 3.

In this example, one or more processing systems 310 are provided coupled to one or more client devices 330, via one or more communications networks 340, such as the Internet, and/or a number of local area networks (LANs). A number of monitoring systems 320, including external monitoring devices and implantable measurement devices, as described above, are provided, with these optionally being connected directly to the processing system 310 via the communications networks 340, or more typically, with these being coupled to the client devices 330.

Any number of processing systems 310, monitoring systems 320 and client devices 330 could be provided, and the current representation is for the purpose of illustration only. The configuration of the networks 340 is also for the purpose of example only, and in practice the processing systems 310, monitoring systems 320 and client devices 330 can communicate via any appropriate mechanism, such as via wired or wireless connections, including, but not limited to mobile networks, private networks, such as an 802.11 networks, the Internet, LANs, WANs, or the like, as well as via direct or point-to-point connections, such as Bluetooth, or the like.

In this example, the processing systems 310 are adapted to receive and analyse subject data received from the monitoring systems 320 and/or client devices 330, allowing subject data to be analysed, and allowing results of the analysis to be displayed via the client devices 330. Whilst the processing systems 310 are shown as single entities, it will be appreciated they could include a number of processing systems distributed over a number of geographically separate locations, for example as part of a cloud based environment. Thus, the above described arrangements are not essential and other suitable configurations could be used.

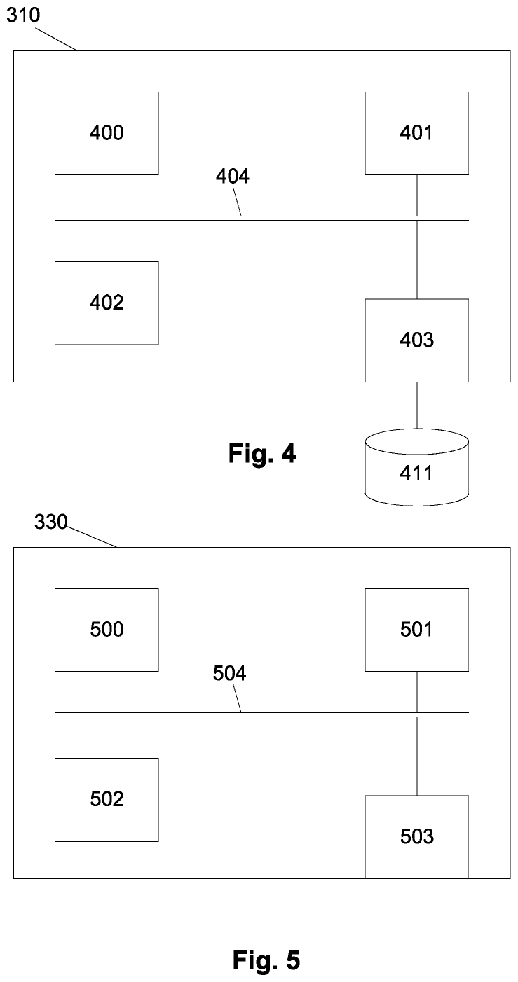

An example of a suitable processing system 310 is shown in FIG. 4. In this example, the processing system 310 includes at least one microprocessor 400, a memory 401, an optional input/output device 402, such as a keyboard and/or display, and an external interface 403, interconnected via a bus 404 as shown. In this example the external interface 403 can be utilised for connecting the processing system 310 to peripheral devices, such as the communications networks 340, databases 411, other storage devices, or the like. Although a single external interface 403 is shown, this is for the purpose of example only, and in practice multiple interfaces using various methods (e.g. Ethernet, serial, USB, wireless or the like) may be provided.

In use, the microprocessor 400 executes instructions in the form of applications software stored in the memory 401 to allow the required processes to be performed. The applications software may include one or more software modules, and may be executed in a suitable execution environment, such as an operating system environment, or the like.

Accordingly, it will be appreciated that the processing system 400 may be formed from any suitable processing system, such as a suitably programmed PC, web server, network server, or the like. In one particular example, the processing system 400 is a standard processing system such as an Intel Architecture based processing system, which executes software applications stored on non-volatile (e.g., hard disk) storage, although this is not essential. However, it will also be understood that the processing system could be any electronic processing device such as a microprocessor, microchip processor, logic gate configuration, firmware optionally associated with implementing logic such as an FPGA (Field Programmable Gate Array), or any other electronic device, system or arrangement.

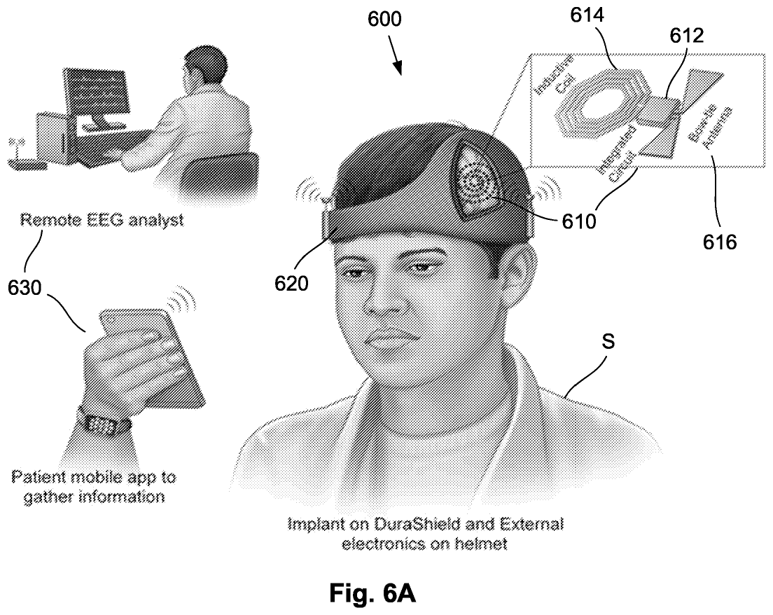

As shown in FIG. 5, in one example, the client device 330 includes at least one microprocessor 500, a memory 501, an input/output device 502, such as a keyboard and/or display, an external interface 503, interconnected via a bus 504 as shown. In this example the external interface 503 can be utilised for connecting the client device 330 to peripheral devices, such as the communications networks 340, databases, other storage devices, or the like. Although a single external interface 503 is shown, this is for the purpose of example only, and in practice multiple interfaces using various methods (e.g. Ethernet, serial, USB, wireless or the like) may be provided.

In use, the microprocessor 500 executes instructions in the form of applications software stored in the memory 501, and to allow communication with one of the processing systems 310 and/or monitoring devices 320.

Accordingly, it will be appreciated that the client device 330 be formed from any suitably programmed processing system and could include suitably programmed PCs, Internet terminal, lap-top, or hand-held PC, a tablet, a smart phone, or the like. However, it will also be understood that the client device 330 can be any electronic processing device such as a microprocessor, microchip processor, logic gate configuration, firmware optionally associated with implementing logic such as an FPGA (Field Programmable Gate Array), or any other electronic device, system or arrangement.

Examples of the processes for monitoring brain activity will now be described in further detail. For the purpose of these examples it is assumed that one or more respective processing systems 310 are servers adapted to receive and analyse subject data, and generate and provide an assessment of brain activity. The servers 310 typically execute processing device software, allowing relevant actions to be performed, with actions performed by the server 310 being performed by the processor 400 in accordance with instructions stored as applications software in the memory 401 and/or input commands received from a user via the I/O device 402. It will also be assumed that actions performed by the client devices 330, are performed by the processor 500 in accordance with instructions stored as applications software in the memory 501 and/or input commands received from a user via the I/O device 502, whilst actions performed by the monitoring devices 320, are performed by the processor 400 in accordance with instructions stored as applications software in the memory 401 and/or input commands received from a user.

However, it will be appreciated that the above described configuration assumed for the purpose of the following examples is not essential, and numerous other configurations may be used. It will also be appreciated that the partitioning of functionality between the different processing systems may vary, depending on the particular implementation.

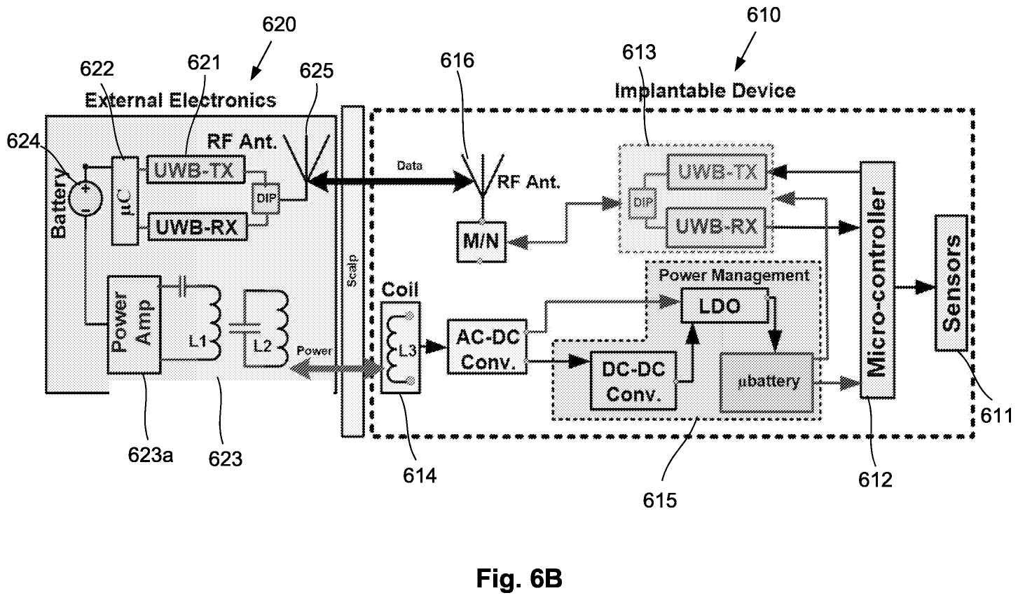

An example of the physical construction of the monitoring system is shown in FIGS. 6A and 6B.

A system 600 for monitoring brain activity of a subject S. The system 600 includes an implantable measurement device 610, an external monitoring device 620 and a client device 630. In this example, the implantable measurement device 610 is embedded on a protector shield of the brain on the subject S. The protector shield is placed on the brain after surgery. Accordingly, the implantable measurement device 610 is able to be in contact to the brain and measure electrical signals of the brain. In this example, the system 600 includes a plurality of implantable measurement devices 610, each operating individually and wirelessly connected to the external monitoring device 620. The external monitoring device 620 is placed in a helmet worn by the subject S, and is in proximity to the plurality of implantable measurement devices 610. As such, the external monitoring device 620 can provide power inductively to each implantable measurement device 610, and transmit and/or receive data to/from each implantable measurement device 610. In this example, the external monitoring device 620 is also in wireless connected to the client device 630, such as a smartphone or a computer. The client device 630 allows the user to view or to further analyse brain activity.

The implantable measurement device 610 has an estimated footprint of approximately 14 mm×7.2 mm and includes: a sensor 611, an electronic measurement processing device 612, an implantable transceiver 613, an implantable antenna 616, and an inductive implantable coil 614. In this example, the electronic measurement processing device 612 and the implantable transceiver 613 takes the form of an implantable integrated circuit. It will be appreciated that the power management module 615 may be partially included in the implantable integrated circuit. The implantable integrated circuit is manufactured using a standard CMOS process, thus the substrate is silicon. The implantable integrated circuit may have a thickness of approximately 0.5 mm with a footprint of 4 mm×4 mm. The thickness of the overall implantable measurement device 610 is approximately 1 to 1.5 mm with a footprint of 14 mm×7.2 mm, including the implantable integrated circuit, a micro-battery, the implantable antenna and the inductive implantable coil. In this example, the implantable integrated circuit is placed on top of the sensor 111. With the given footprint, there are four electrodes placed under the implantable integrated circuit. This allows ECoG signals to be measured and in one example allows these to be measured with a temporal resolution up to approximately 10 kHz and a spatial resolution of 1 cm whereas EEG is generally approximately 512 Hz with a spatial resolution that is difficult to determine.

Additionally, the implantable antenna 616 and the inductive implantable coil 614 are manufactured as a flexible patch like the one used for RFID antennas in the industry (inlay), and the implantable integrated circuit is bonded directly to the antenna 616 and coil 614 inlay without any extra package. As a result, the implantable measurement device 610 is able to remain generally small and flexible.

The external monitoring device 620 includes an external transceiver 621, an electronic monitoring processing device 622 and an inductive external coil 623. In this example, the external monitoring device 620 includes an energy storage unit 624. Due to the size of the helmet and processing requirements, there are a number of power supply options available including conventional lithium cells. The external monitoring device 620 in the helmet are similar to the implantable measurement device 610 described above, but with considerably relaxed design constraints in terms of size, weight and power consumption. The external monitoring device 620 acts as a base station with much more efficient design parameters, while the implantable measurement device 610 are like mobile terminals with limited design features such as gain, efficiency, size, power consumption etc., due to the application environment and the physical constraints on size and weight.

The external monitoring device 620 acts as a communication gateway between the implantable measurement device 610 and the client device(s) 630. The external monitoring device 620 requires careful design techniques to achieve appropriate performance in terms of effectiveness and sensitivities in order to detect the data from implantable measurement device 610 and supply the power to the implantable measurement device 610. The external monitoring device 620 does not interfere with the biological systems being measured. The external monitoring device 620 may also be used to configure the implantable measurement device 610 and set up the parameters for the operation of the implantable measurement device 610.

In this example, the client device 630 is provided for patients and medical practitioners to view event information of the subject. The client device 630 is in wireless communication with the external monitoring device 620 using a frequency band that does not interfere with the communication between the external monitoring device 620 and the implantable measurement device 610. The client device 630 can be a computer, tablet or smartphone with communication and display capabilities.

As mentioned, the implantable measurement device 610 includes an implantable transceiver 613 that operates in the ultra-wideband (UWB) frequency spectrum ranging from 3.1 to 10.6 GHz. Similarly, the external transceiver 621 also operates in the same frequency when in communication with the implantable transceiver 613. The transceivers are configured such that the transmitter and the receiver share the same antenna. In this example, an IR-UWB system architecture has been targeted using On-Off Keying (OOK) or the Binary Phase Shift Keying (BPSK) modulation schemes, providing uplink data rate of 500 Mbps and downlink data rate of 100 to 200 Mbps.

According to the above, the implantable antenna 616 and the external antenna 625 are UWB antennas. In one example, the antennas have the phase centre and voltage standing wave ratio (VSWR) being constant across the whole bandwidth of operation, which can be achieved by setting the antenna resonating frequency above the operating frequency band.

Throughout the proceeding paragraphs, optimisation examples of an implantable antenna of the present invention will be described in further details.

It will be appreciated that the performance of RF antennas are determined by the amount of signal power or radio waves presented at the antenna input. The ratio of reflected radio waves to the absorbed radio waves measured in decibel (dB) is commonly referred to as the return loss. Return Loss in dB is 10*log₁₀ (Pᵢ/Pᵣ), where Pᵢ is the incident power and Pᵣ is the reflected power. S₁₁ in dB is 20 log₁₀ |Eᵣ/Eᵢ|=10 log₁₀(Pᵢ/Pᵣ), where Eᵣ is the reflected field and Eᵢ is the incident field. Hence return loss=−S₁₁. As a general principle, it is preferred that the return loss is expected to be below about −10 dB.

Another important measure of the antenna performance is the voltage standing wave ratio, is the parameter that determine if the antenna is properly matched to the radio. It is also a function of the reflection coefficient that describe the amount of power refracted from the antenna.

$$VSWR = \frac{1 + \vert Γ \vert}{1 - \vert Γ \vert} ;$$

where Γ is the reflection coefficient. In the case of the bow-tie antenna it is important to keep the VSWR below 2 dB. The characteristics impedance is matched to 50 ohm.

A bow-tie radio frequency antenna can deliver a high data rate as a result of its wideband width nature and is thus one example of an antenna configuration in a preferred embodiment of the present invention. Without limitation, a suitable antenna would operate in the frequency range of about 7.5 GHz to about 9.5 GHz and would have a bandwidth of about 1.3 GHz. The design and fabrication of an antenna for an implant requires the selection of suitable materials that facilitate biocompatibility and efficient performance inside or around human tissue.

It will be appreciated that one skilled in the art would be able to select a suitable material for the fabrication of the implantable antenna used in the implantable transceiver. The requirements of the implantable antenna generally include being flexible, biocompatible and having the capability of being miniaturized. A suitable material includes the use of copper-clad laminated composites having a flexible polymeric substrate (polyimide, polyethylene etc). An example of a commercially available and preferred material includes DuPont Pyralux®, which comprises a flexible polyimide film substrate with copper foil on one or both sides of the substrate as a patch. Substrates with higher dielectric constants yield better performance and are thus preferred.

As noted in the preceding paragraphs, miniaturization of the implantable antenna is required without affecting its overall efficiency in the implanted environment. One method of miniaturisation is achieved by combining a bow-tie structure with a folded dipole structure. In this embodiment, the bow-tie antenna achieves wider bandwidths while the folded dipole permits a wavelength extension, thus leading to optimisations and an overall reduction in an implantable footprint.

The effects of human tissue on the transmission from the transceiver must also be considered. Brain tissue is comprised white matter and grey matter whose dielectric properties are measured to be at frequencies ranging from 0.01-10 GHz.

The link budget is an estimated amount of gains and losses from the transmitter through the human tissue or free space to the receiver in a communication system.

The average transmitted power (PTX)=EIRP*BW*TX_Ratio,

FCC mandate EIRP=−41.3 dBm/MHz, bandwidth (BW)=600 MHz and TX_Ratio is assumed to be 4 dB.

Antenna gain for the transmitter and receiver is assumed to be 0 dB, only one antenna is used. Hence GTX=GRX.

The path loss (PL)=20 log(4 πd/λ) where d is the distance between transmitter and receiver. Lambda λ is the wavelength.

The receiver power (PRX)=PTX*antenna gain/PL

Noise Power (Po) or Thermal Noise floor=k*T*BW

K=Boltzmann constant, T=Room temperature, BW=Information bandwidth

Sensitivity (S)=Noise floor+NF+C/N (Carrier to Noise Ratio)

Link margin (LM)=PRX−S

Assuming On-Off-Keying with signal to noise ratio (SNR) of 11.5 dB.

Since SNR=Eb/No and No=kT where k is Boltzmann constant and T is temperature in kelvin.

The Energy per bit (Eb in Joule/bit).

The channel capacity (C in bits/sec) for system limited by thermal noise is given by

$$C = B * log 2 ( 1 + SNR )$$

Energy per bit required for the system may be calculated

$$( Eb ) sys = ( P\_TX + P\_RX ) \text{/} R$$

The implantable measurement device 610 is inductively powered by the external monitoring device 620 via the inductive implantable coil 614 and the inductive external coil 623. The inductive external coil 623 includes a power amplifier 623a, a driver coil L1 and a repeater coil L2. In this example, the inductive external coil 623 operates at 13.56 MHz frequency and delivers power to the inductive implantable coil 614 for a nominal coupling distance of up to 10 cm at approximately 35 percentage of efficiency. In this example, the inductive implantable coil 614 is directly connected to an AC-DC converter where the energy is converted to a DC voltage and further regulated through a DC-DC converter and low drop-out regulator before it is stored on a charge storage such as a super-capacitor or a micro-battery. The charge storage distributes power within the implantable measurement device 610.

A mutual inductance is produced as a result of current flowing in primary coil that is induced to another secondary coil opposite or adjacent as a voltage. The mutual inductance unit of measurement is Henry. In this case the mutual inductance is inversely proportional to distance. As the distance increases mutual inductance decreases. The implantable coil may be constructed from the following design parameters; outer diameters (Do), inner diameter (Di), coil width (w), spacing between coil (s) and number of turns (N). These parameters are chosen through design calculation as follows; Do=5.2 mm, Di=2.5 mm, w=150 μm, s=150 μm and N=5. Implantable coil for energy harvester, the coil is made of copper conductor on a polyimide substrate, the crossing arm is connected with two via holes of about 0.124 mm diameter.

In preferred embodiments, the implantable coil is fabricated using a copper conductor on a polyimide substrate, the crossing arm is connected with two via holes of about 0.124 mm diameter.

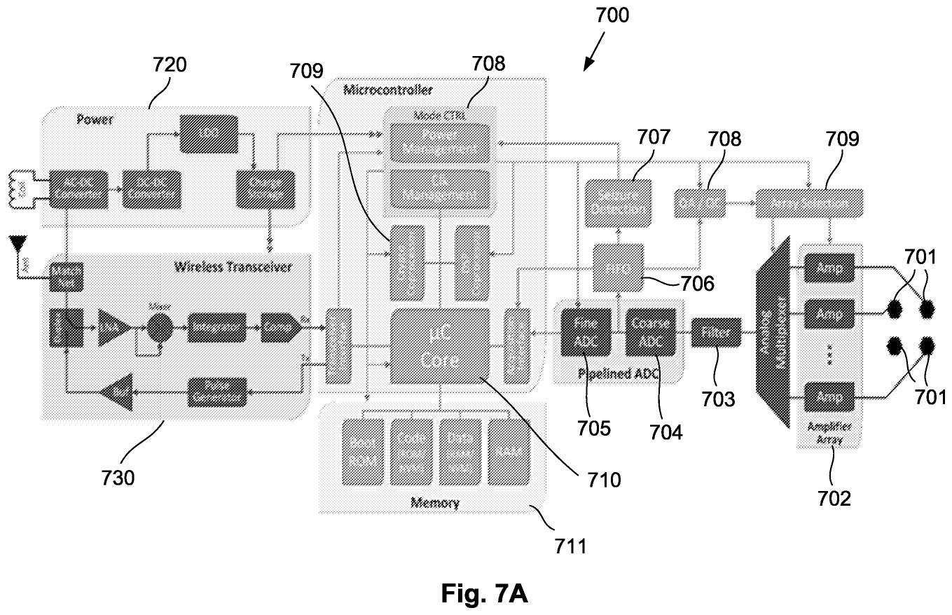

Specific functional features of an example of the implantable measurement device 700 are shown in FIGS. 7A to 7C.

The implantable measurement device 700 includes four electrodes 701 for measuring electrical signal of the brain. Each electrode 701 is coupled to a respective amplifier in the amplifier array 702 to amplify measurement signals. The amplifier array 702 is coupled to a sampling module via a filter 703, which optionally filters the measurement signals. The sampling module includes a coarse analog-to-digital converter (ADC) 704 and a fine ADC 705, which can be implemented as physically separate ADCs, using a single ADC operating in accordance with different operating parameters, or using a two stage ADC with the first stage providing coarse filtering. This allows for sampling the measurement signal in different sampling rates and/or sampling resolutions. A temporary memory 706, such as a FIFO memory, is coupled to the sampling module to temporarily store the sampled data. The seizure detection module 707 reads the sampled data and determines if the sampled data is indicative of seizure. The sample data is also read by a data quality module 708 which determines the inaccurate amplifiers by performing one or more data quality tests. The determination is fed to an array selection module 709 to ensure the quality of the measurement signals and sampled data.

An output of the seizure detection module 707 is used by a mode control module 708 that controls operation modes of the implantable measurement device 700. The mode control module 708 controls the modules to be active or inactive in different modes. The mode control module 708 is also connected to an encryption module 712 and a core processing module 710. The core processing module 710 is further connected to a central memory module 711. A power module 720 and an implantable transceiver 730 are also connected to the mode control module 708. The power module 720 provides power to the implantable measurement device 700 whereas the implantable transceiver 730 transmits and receives to and from an external monitoring device.

In this example, the implantable transceiver 730 further includes an implantable receiver 731, an implantable transmitter 732 and an implantable antenna 733 for wirelessly communicate with the external monitoring device. The implantable transceiver 730 may operate in a receiving mode, a transmitting mode and a waiting mode. When in the waiting mode, the implantable transceiver 730 receives an incoming signal via the implantable antenna 733. A wake-up module 734 determines if the incoming signal is in a predetermined transmission band.

As shown in FIG. 7B, if the incoming signal is in the predetermined transmission band, a wake-up module 734 activates the implantable receiver 731 to receive the incoming signal, so that the implantable transceiver 730 is operating in the receiving mode. If the incoming signal is not in the predetermined transmission band, the implantable transceiver 730 remains in the waiting mode, so that minimal power is consumed.

The implantable transceiver 730 switches to operate in the transmitting mode when the incoming signal is in the predetermined transmission band and is determined to be a transmission request by the mode control module 708. As shown in FIG. 7C, when in transmitting mode, the implantable transmitter 732 is active to transmit data via the implantable antenna 733. During data transmission, the wake-up module 734 and the implantable receiver 730 are deactivated, since no incoming signal is expected. Once the transmission is completed, the implantable transceiver 730 changes its mode to the waiting mode.

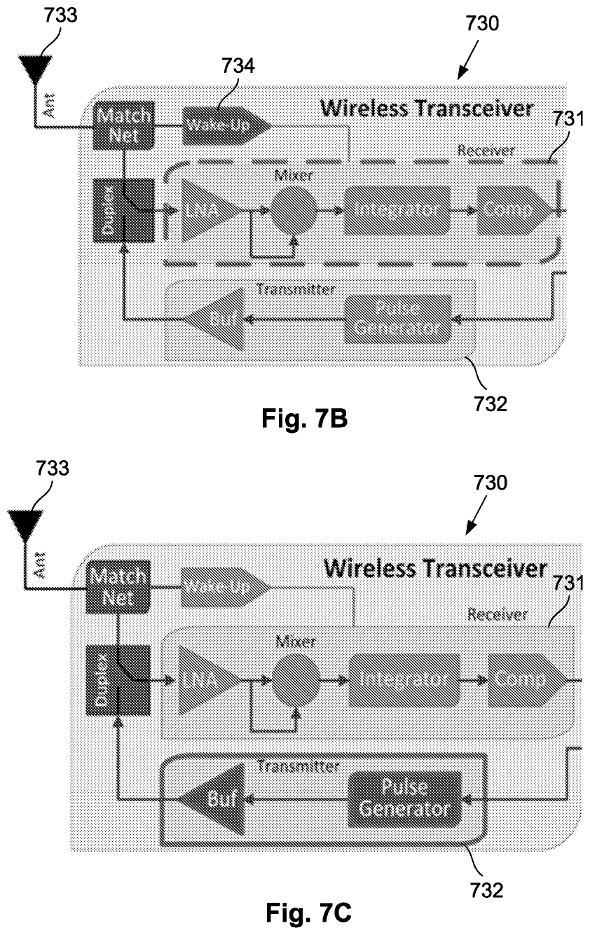

An example of an overall operation of the implantable measurement device 700 will now be described with reference to FIG. 8.

In this example, the implantable measurement device is operable in a standby mode and an active mode. At step 810, the implantable measurement device is in a standby mode monitoring brain activity of a subject. When an event of the brain activity is detected at step 820, the implantable measurement device switches to operate in the active mode at step 830. The implantable measurement device remains in the standby mode if no event is detected. The implantable measurement device switches from the active mode to the standby mode when normal brain activity is detected at step 840.

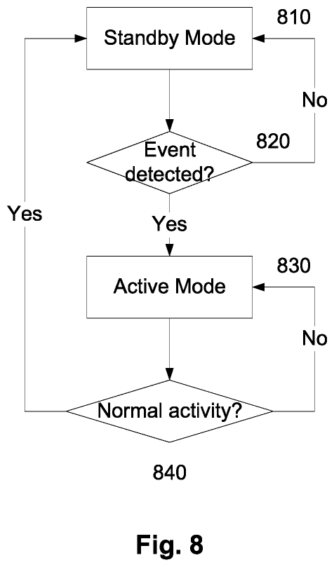

An example of a detail operation of the implantable measurement device will now be described with reference to FIGS. 9A and 9B.

According to the above, the implantable measurement device is operable in the standby mode. When in the standby mode, at step 900, the array selection module 709 selects a reduced number of amplifiers. At step 905, measurement signals are received from the selected amplifier(s) 702, being optionally filtered at step 910 by the filter 703, to remove noise. The filtered signals are then sampled by the coarse ADC 704 at a low sampling rate and/or a coarse sampling resolution at step 915. The sampled signal is temporarily stored in a FIFO memory 706 at step 920. Subsequently, at step 925, an event detection module 707 analyses the sampled signal. At step 930, if it is determined that the sampled data is indicative of an event of the brain activity the implantable measurement device is switched to operate in active mode. Otherwise, the data quality module determines inaccurate or faulty amplifiers at step 935 before returning back to step 900 where the implantable measurement device continues to operate in the standby mode.

In the active mode, at step 940, the array selection module 709 selects all available amplifiers. In one example, available amplifiers can be the amplifiers connected to the electrodes with accurate sensing or all active amplifiers, depending on the preferred implementation. At step 945, the measurement signals are received from all available amplifier(s) and transferred to the filter 703, allowing the measurement signals to be filtered at step 950 to remove noise. The filtered signals are sampled by the fine ADC 705 at a high sampling rate with a fine sampling resolution at step 955. The sampled signal can be temporarily stored in a FIFO memory at step 960. Subsequently, at step 965, the sampled signal is processed by a core processing module 710 to generate an event data, which is then stored in a central memory module 711. The event data may be further encrypted before storing in the central memory module for transmission. Alternatively, the event data may be read from the central memory module and encrypted before transmission.

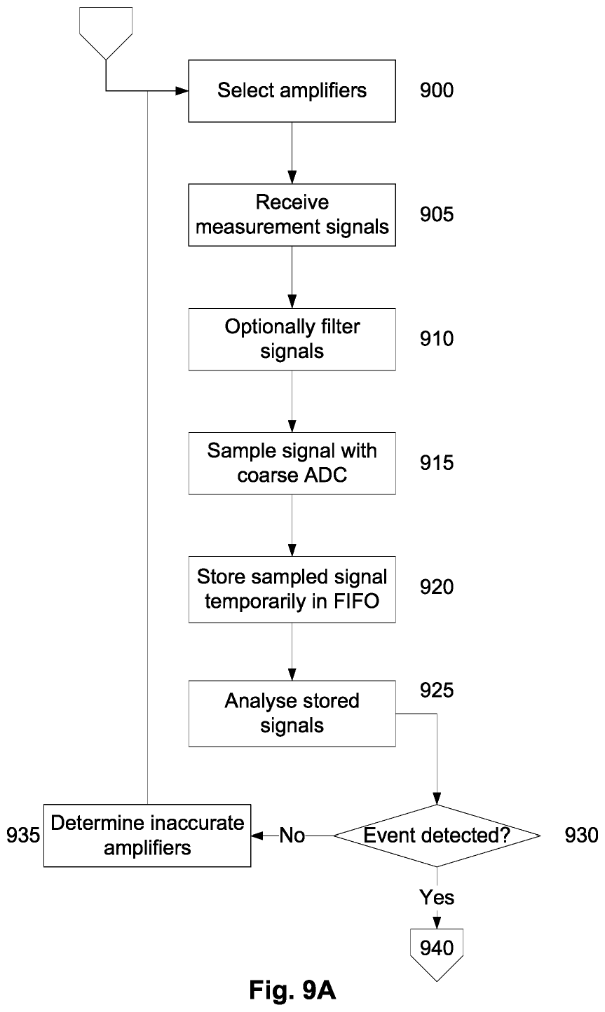

An example of an operation of the analysing system of FIG. 3 will now be described with reference to FIG. 10.

At step 1000, the external monitoring device 620 requests event data from the implantable measurement device 610, causing the implantable measurement device 610 to retrieve the event data from the memory module 711 and encrypt the event data using the encryption module 712 at step 1010. As previously discussed, this can be performed using a session key exchanged with the external monitoring device 620, or could be performed using an external monitoring device 620 public key.

At step 1020, the encrypted event data is uploaded to the external monitoring device 620, which uses this to generate subject data. This will typically involve decrypting the event data and optionally adding information, such as a subject identifier or similar, before the subject data is uploaded to a server 310 at step 1030. It will be appreciated that this might also involve the subject data being encrypted in a similar manner. The subject data can be uploaded to the server 310 directly, or could be transferred to the client device 330, which in turn transmits the subject data to the server 310, based on information received from the external monitoring device 320.

At step 1040, the server 310 analyses the subject data, for example, by calculating one or more metrics, and then analysing the metrics to generate activity data indicative of the monitored brain activity at step 1050. In one example, the indicator is calculated by comparing the received data with pre-stored patterns. In another example, the indicator is calculated by inputting the received data to a machine learning algorithm or neural-network.

At step 1060, the activity data can be provided to a client device 330, such as a computer, allowing the client device to display an activity indicator, which can include details of the event, such as an event type, time, duration or severity. This allows the details of the event to be reviewed by a medical practitioner, allowing interventions to be performed as required. In another example, the indicator is reported on a smartphone 330 of a caretaker or the subject, allowing this to act as a seizure alert.

The client device may show one or more of the following to a user:
A dashboard summary of major measurements;
A real-time EEG and temperature;
system health diagnostics; and/or
other health records relevant to patient being monitored.

While not limited, in preferred embodiments of the present invention, the client device may include one or more of the following: a computer, a handheld smart device (e.g. mobile phone) and/or a suitable immersive reality device (e.g. a Microsoft HoloLens).

As discussed previously, the client device is in wireless communication with the external monitoring device using a frequency band that does not interfere with the communication between the external monitoring device and the implantable measurement device.

There are a number of networking technologies that may be considered including Wi-Fi, Bluetooth, and mobile data networks (e.g. 3/4G, 4G LTE). It is also envisaged that a combination of these wireless technologies may be used to provide a failover stack, whereby the external monitoring device electronics will select the best available networking option for communication. The selection will need to consider the availability of each networking option and the appropriateness for the intended application. An example of this is communicating with a mobile device as opposed to a central server. For the mobile device, Bluetooth would be the preferred option due its proximity to the external monitoring device. In contrast, when communicating with a central server, Bluetooth may not be a viable option due to the limited range and no access to the broader network.

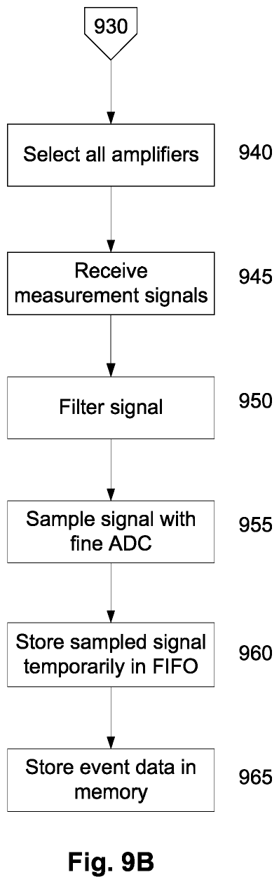

The data that is collected and processed the device of the present invention may be further processed by the external monitoring device prior to its transmission to the user. The nature of the user interface will depend on the user and it is envisaged that a range of user interface settings will be available to suit the differing needs and data comprehension abilities of the patients and medical specialists. A graphical interface which presents the raw data in an easily understood fashion is the goal. A service-based architecture will allow multiple stakeholders to subscribe to information streams for different patients. By using a service-based approach, the subscribers are largely decoupled from the data stream. This allows interfaces to be developed for different platforms; such as mobile devices, desktop computers, analytics services, etc.; more easily. Input from a range of potential users will be sought during the user interface design, whereby a proposed architecture can be seen in FIG. 11.

As shown, the primary flow of information from the external monitoring device incorporated into the user's helmet or headgear occurs via a wireless network connection to a services API. This allows processing of the data or events, storage of the data/event, and notification to subscribed users. Subscribed users are permitted to view the data and conduct local analysis or monitoring.

In a preferred embodiment, all requests for data are made through the external monitoring device.

In the following paragraphs, data processing and capture algorithms included in preferred embodiments of the present invention will be described in further detail. It is envisaged that one or more signal processing algorithms will be included as a system-on-chip (SoC) design and included with the implantable measurement device, to facilitate seizure detection and/or prediction. To realize complex algorithms within the limited on-chip computational capacity, dedicated signal processing hardware blocks will be included in the system to accelerate specific calculations.

One or more digital filters are incorporated into the implantable measurement device as dedicated hardware to be used for on-chip signal processing. These filters are used to separate the EEG signal monitored by the device into different frequency band. These frequency bands are defined as Delta (1-4 Hz), Theta (4-8 Hz), Alfa (8-13 Hz), Beta (13-20 Hz) and Gamma (20-55 Hz) which show different activity levels of the brain. It will be appreciated that the frequency bands configured for the various filters is not overly limited and can be configurable to any cut-off frequency in the interest of flexibility. In one example, the high frequency band filter may have a cut-off frequency of about 150 Hz.

For the detection of seizures from the monitored EEG signals, machine learning algorithms can be implemented as a dedicated hardware and/or software as part of the System-on-Chip (SoC) design.

As will be appreciated, EEG recordings provide significant information relating to brain activity at any given time and is used to detect the onset of seizure events in real-time.

A Fast Fourier Transformation (FFT) can be used to examine the frequency composition of EEG signal measured by the implantable measurement device. The change of the FFT with time is represented by using Short Time Fourier Transformation (STFT) which is the FFTs of consecutive short time windows. It is envisaged that a time window of about 512 samples (1 second) may be used.

As discussed previously, the one or more electronic measurement processing devices of the implantable measurement includes a data quality module which evaluates data quality of each individual channel in real-time. These modules ensure there is a high degree of confidence and reliability in the seizure detection and predication outcome. At a base level application, the data quality module may assess 1 minute of EEG data provided from the FIFO memory using one or more data quality tests (DQTs). The results from these tests are then combined and classified using, in a preferred embodiment, fuzzy logic.

One possible data quality test (DQT) from an EEG signal is performed on the amplitude, or intensity, of the voltage at the peaks in the signal data. This ‘spike’ is a good measure of data quality and is used to ensure the mean and standard deviation of measured voltage values during a predetermined time window are within reasonable and expected limits for during normal physiological activity, such as those measure during normal brain activity.

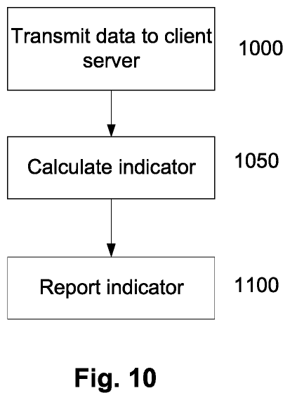

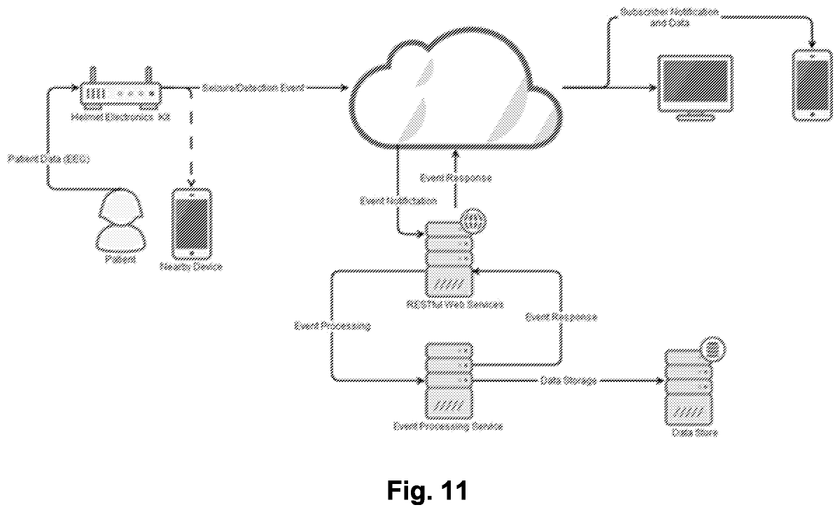

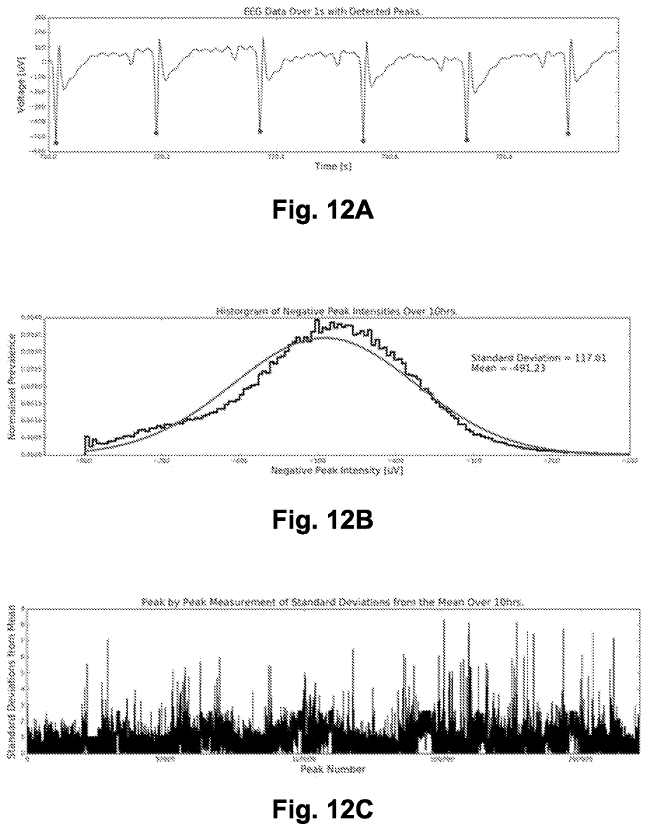

Using this first DQT as an example, the lower peaks are detected from the EEG data. As an example, this analysis is highlighted in FIG. 12A. The negative peaks are then subsequently assessed as they may be more prominent in intensity than the positive peaks. These peaks are also the first major voltage spike after a relatively low voltage period, and therefore are deemed more reliable. It should be appreciated that this determination of mean and standard deviation can be implemented via hardware on the implantable measurements device and/or via the external monitoring device. However, it is preferred that the determination is made via the data quality module included in the implantable measurement device.

The intensity of the determined peaks are defined as the voltage amount from the ground of about 0 volts. This is an accurate measurement assuming there is no signal drift. In some instances, it may be more complete to take the intensity from the moving average of the signal to ensure unexpected intensities are detected regardless of the actual data quality, which will further inform the fuzzy logic system.

A histogram displaying the distribution of peak intensities is then created to show the expected intensity values. This is then fitted with a Gaussian distribution providing a mean and standard deviation, as shown in FIG. 12B. This distribution allows an individual peak intensity to be compared to the expected range of values as seen in FIG. 12A—each data point represents a detected peak, with its difference from the average intensity measured in standard deviations.

Another DQT, which may be used alone or in conjunction with any other DQT, may be performed by using the distance between each EEG signal peak that is measured via each electrode. This can be measured and compared across the minute of data in the FIFOs storage to previous measurements.

Using the known time locations of the peaks, the difference in distance between each peak is stored. Once stored, an average EEG wave is generated to give the ideal wave for that 1 minute time period, an example of 10 minutes can be seen in FIG. 12A. This average EEG wave can then be compared to a control EEG wave, which may include either a control preloaded into the memory or could also include a comparison against different channels. Additionally, the distances between each peak is plotted as a histogram. The histogram then is curve fitted using multiple Gaussian models as in FIG. 12C. The parameters for the fitted Gaussian models are then received by the fuzzification system and the data is classified.

In a preferred embodiment of the present invention, a fuzzy-logic system is adopted to allow the device to be capable of classifying the degree of confidence and reliability in the automated data quality tests discussed in the preceding paragraphs. The fuzzy-logic system assesses multiple DQTs consecutively, which results in a continuous flow of QA/QC. As discussed, the DQTs such as peak-to-peak distance and amplitude fluctuations are the main focus from the raw EEG signals, thus the parameters used in the fuzzy-logic functions can be tailored for each of these data quality tests.

In this embodiment, the fuzzy-logic system receives mean and standard deviation values obtained from Gaussian fittings for each of the data quality tests. There could be multiple Gaussians present and thus an equivalent number of means and standard deviations returned. The fuzzy-logic system then assesses data from the array of the DQTs and determines whether it is within a suitable range of a mean, dependent on standard deviation.

Actions are dependent on the fuzzification of data and can be determined through a membership function for the parameters contributing to data quality table. The table represents two parameters which compare their closeness to a mean position by standard deviation, a.

| | Parameter 1 | | | | |
|---|---|---|---|---|---|
| Correlation | <1σ | 1σ-2σ | 2σ-3σ | 3σ-4σ | >4σ |
| | Parameter 2 | | | | |
| <1σ | Do | Do | Check other | Monitor | Monitor |
| | nothing | nothing | electrodes | | |
| 1σ-2σ | Do nothing | Check | Check | Monitor | Turn on |
| | | other | other | | seizure |
| | | electrodes | electrodes | | detection |
| 2σ-3σ | Check | Check | Monitor | Turn on | Turn on |
| | other | other | | seizure | seizure |
| | electrodes | electrodes | | detection | detection |
| 3σ-4σ | Monitor | Monitor | Turn on | Turn on | Faulty |
| | | | seizure | seizure | Electrode |
| | | | detection | detection | |
| >4σ | Monitor | Turn on | Turn on | Faulty | Faulty |
| | | seizure | seizure | Electrode | Electrode |
| | | detection | detection | | |

When data falls outside the expected range, it is categorised as unreliable data and/or representative of a possibly faulty electrode. The advantage of using a fuzzy based system, as part of the QA/QC, is its ability to categorise DQT values into their respective levels of confidence and reliability, without the need for extensive or difficult computations.

In a preferred embodiment of the present invention, the one or more electronic measurement processing devices includes one or more configurable embedded algorithms involved in the detecting and predicting the seizure event. This includes one or more configurable embedded signal processing and feature extraction algorithms, such as standard deviation, peak-to-peak distance, peak amplitude, integration, differentiation, and machine learning (ML) algorithms. The signal processing and feature extraction algorithms are reconfigurable in terms of width of time window that is being evaluated, amplitude resolution (number of bits per sample), some limits and set parameters to use in feature extraction. The configurability of the machine learning algorithm enables selecting which extracted features to use and the weights of the classification algorithm. This configurability provides adaption to meet specific operational context, compensate variations due to instalment, individualisation of the device and also repurposing the device for different applications such as detecting different phenomena in EEG signal or other bio-signals.

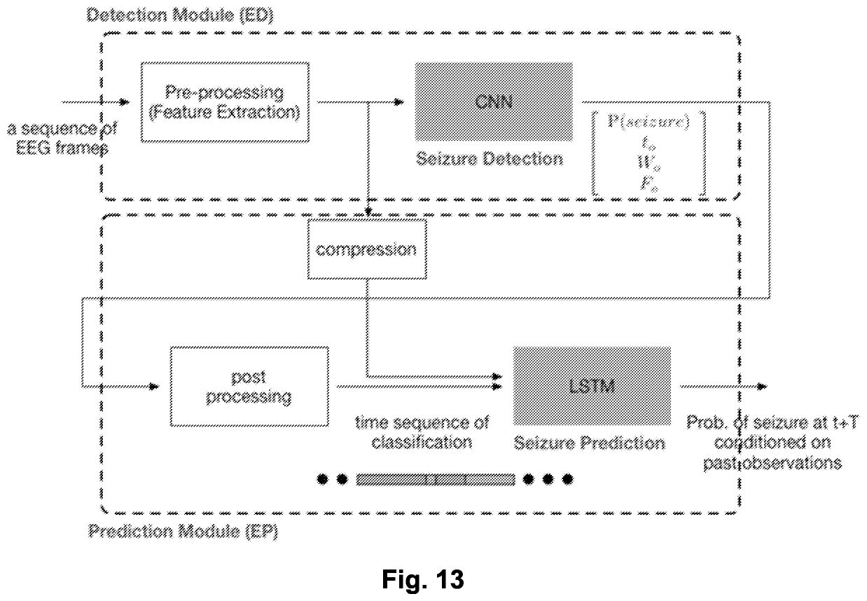

In preferred embodiments, on-board components are called on to perform each function as the seizure detection module (ED) and the seizure prediction module (EP), respectively. The ED module classifies whether they contain seizures or not, and, if exists, identifies the location of seizures in the EEG frame. The EP module consumes the time sequence of the output of the ED module and produces the conditional probability of future seizure occurrence based on past observations. FIG. 13 shows the structure of the seizure detection (ED) and the prediction module (EP).

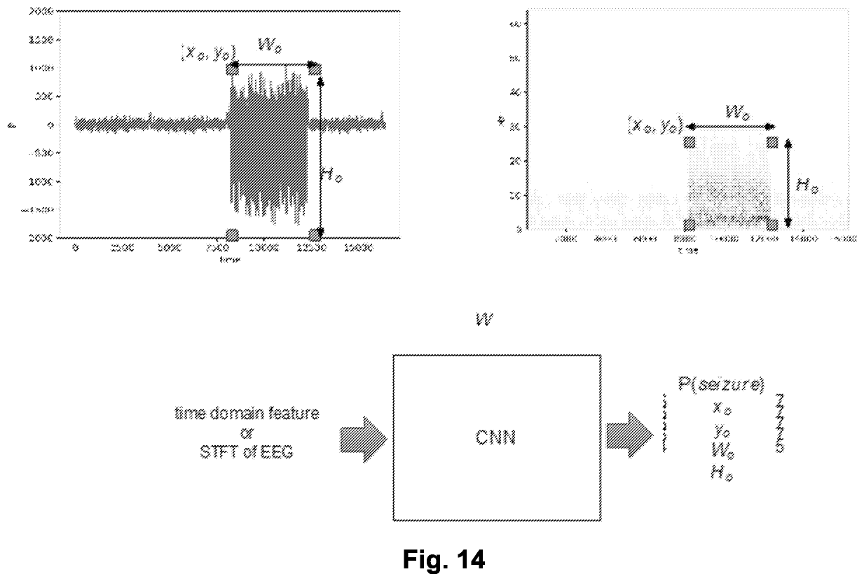

As shown in FIG. 14, the seizure detection (ED) module classifies the occurrence of seizures by applying convolutional neural networks (CNN). CNNs are an example of emerging deep learning technology that is known in the art to show excellent performance in image classification with minimal pre-processing overhead. The output of the ED module is the probability of containing seizures and the location of the seizure, if exists. Here, the location of seizure is presented by a vector (x₀, y₀, W₀, H₀) where each element is described in FIG. 14.

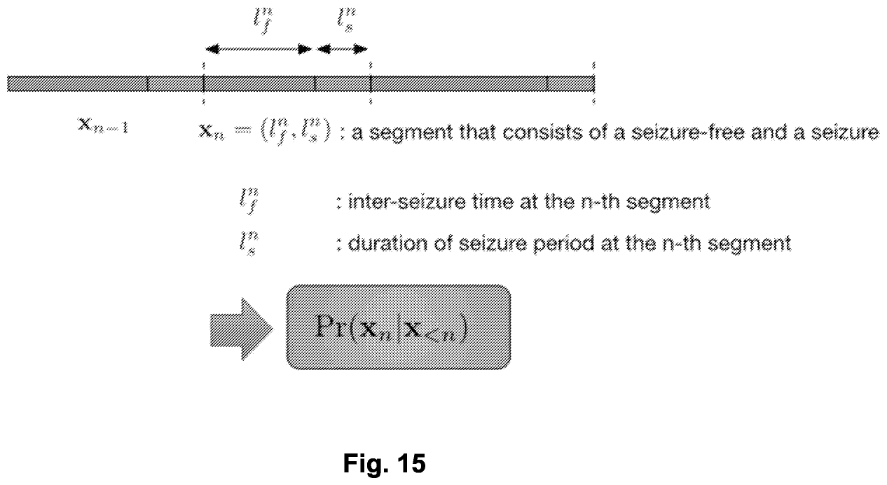

The prediction module (EP) determines the probability of future seizure occurrence based on past observations made by the implantable measurement device. As shown in FIG. 15, it utilises a sequence of outputs from the ED module to evaluate the probability, in which the outputs of the ED module are reconstructed to be a sequence of a pair (start time, duration) of a seizure. As the expected output is the conditional probability based on input sequences, a recurrent neural network (RNN) is employed for the EP module development. In one example, the seizure prediction problem is reformulated as a sequence estimation problem by considering the prediction as the estimation of the next seizure occurrence based on a sequence of past seizure observations. Specifically, a long-short term memory (LSTM) is employed for the implementation of the RNN.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers. As used herein and unless otherwise stated, the term “approximately” means ±20%.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

It will of course be realised that whilst the above has been given by way of an illustrative example of this invention, all such and other modifications and variations hereto, as would be apparent to persons skilled in the art, are deemed to fall within the broad scope and ambit of this invention as is herein set forth.

1) A system for monitoring brain activity of a subject, the system including:
a) at least one implantable measurement device including:
i) a sensor configured to measure electrical activity in the brain;
ii) one or more electronic measurement processing devices configured to:
(1) receive measurement data from the sensor;
(2) use the measurement data to determine if the brain activity of the subject is indicative of an event; and
(3) generate event data indicative of the brain activity associated with the event; and
iii) an implantable transceiver configured to transmit the event data;
iv) an inductive implantable coil configured to inductively receive power; and,
b) an external monitoring device including:
i) an external transceiver configured to receive event data from the implantable measurement device;
ii) an inductive external coil configured to inductively transmit power to the implantable measurement device; and,
iii) one or more electronic monitoring processing devices configured to:
(1) generate subject data; and,
(2) transfer the subject data to one or more analysis processing devices for analysis.

2) The system according to claim 1, wherein the at least one implantable measurement device is at least one of:
a) placed under a skull of the subject;
b) placed on dura mater of the subject;
c) at least partially embedded in a protector shield protecting the brain; and/or
d) further comprises at least one of:
i) a temperature sensor, the measurement data being indicative of a temperature;
ii) a pressure sensor, the measurement data being indicative of a pressure; and,
iii) a pH level sensor, the measurement data being indicative of a pH level;
iv) one or more amplifiers, each amplifier being configured to amplify measurement signals from a respective electrode;
v) a sampling module configured to sample a measurement signal from the sensor;
vi) a temporary memory for storing the measurement data;
vii) a data quality module for identifying inaccurate sensing from an electrode;
viii) a data memory for storing the event data;
ix) an encryption module for encrypting the event data before transmitting;
x) an implantable energy storage unit; or
e) has a physical footprint of approximately 14 mm×7.2 mm.

3-5. (canceled)

6) The system according to claim 2, wherein the sensor:
a) includes one or more electrodes, wherein, optionally, the sensor includes four electrodes; and/or
b) is mounted on the one or more electronic measurement processing devices.

7) (canceled)

8) The system according to claim 2, wherein the one or more electronic measurement processing devices have a physical footprint of approximately 4 mm×4 mm.

9) (canceled)

10) (canceled)

11) The system according to claim 2, wherein the at least one implantable measurement device includes an array selection module for selectively activating the one or more amplifiers.

12) (canceled)

13) The system according to claim 6, wherein the sampling module includes at least one of:
a) an analog-to-digital converter; and,
b) a coarse analog-to-digital converter and a fine analog-to-digital converter.

14) (canceled)

15) (canceled)

16) The system according to claim 6, wherein the event includes a seizure.

17) (canceled)

18) The system of claim 8, wherein the data quality module is configured to perform at least one of:
a) a first data quality test; and,
b) a second data quality test.

19) The system of claim 8, wherein the first data quality test includes at least one of:
a) determining mean and standard deviation values from peak amplitude values in EEG data measured from the electrode; and,
b) determining whether the determined mean and standard deviation values are within a predetermined range with a fuzzy-logic system.

20) (canceled)

21) (canceled)

22) The system according to claim 2, wherein the second data quality includes determining a distance between consecutive EEG signal peaks measured via the electrode and calculating an average EEG wave.

23) The system according to claim 11, wherein at least one of:
a) the second data quality test further includes comparing the average EEG wave of the electrode against a control EEG wave;
b) the control EEG wave is preloaded onto a memory of the implantable measurement device; and,
c) the control EEG wave includes an average EEG wave calculated based on measurements of one or more further electrodes.

24) (canceled)

25) (canceled)

26) (canceled)

27) (canceled)

28) The system according to claim 21, wherein at least one of:
a) the implantable transceiver includes an implantable antenna; and,
b) the external transceiver includes an external antenna.

29) (canceled)

30) The system according to claim 13, wherein the implantable energy storage unit is at least one of:
a) a micro-battery; and,
b) a three-dimensional micro-battery.

31) (canceled)

32) (canceled)

33) (canceled)

34) The system according to claim 1, wherein the external monitoring device:
a) includes at least one of:
i) an external power amplifier inductively connected to the inductive external coil;
ii) an external energy storage unit providing energy to the inductive external coil; wherein, optionally, the external energy storage unit is at least one of: a micro-battery; and a three-dimensional micro-battery; and/or
b) is placed in a headgear wearable by the subject.

35) (canceled)

36) (canceled)

37) (canceled)

38) (canceled)

39) The system according to claim 2, wherein the one or more electronic measurement processing devices configured to be at least one of:
a) operable in a standby mode and an active mode depending on whether an event is occurring;
b) operable in a receiving mode and a transmitting mode according to a transmission request; and
wherein, optionally, when in the standby mode, the one or more electronic measurement processing devices are configured to:
a) identify inaccurate sensing from an electrode;
b) select one or more amplifiers based on identified inaccurate sensing;
c) receive measurement signals from the selected amplifiers;
d) filter the measurement signals;
e) sample the measurement signals at a low sampling rate and/or a coarse sampling resolution; wherein, optionally, wherein the low sampling rate is approximately 512 Hz, and the coarse sampling resolution is 8-bit; and
f) store sampled signal data in a temporary memory.

40) (canceled)

41) The system according to claim 2, wherein the one or more electronic measurement processing devices are at least one of:
a) configured to:
i) at least partially analyse the sampled signal data;
ii) determine if an event is occurring in accordance with results of the analysis; and,
iii) if an event is occurring, switch to the active mode; and,
b) determine if an event is occurring by at least one of:
i) analysing one or more parameters derived from the sampled signal data;
ii) comparing the sampled signal data to previous sampled signal data; and,
iii) using machine learning techniques.

42) (canceled)

43) (canceled)

44) The system according to claim 16, wherein, when in the active mode, the one or more electronic measurement processing devices are configured to:
a) select all available amplifiers;
b) receive measurement signal from the selected amplifiers;
c) filter the measurement signal;
d) sample the measurement signal at a high sampling rate and/or a fine sampling resolution; and
e) store event data including sampled signal data in a data memory;
wherein, optionally, the high sampling rate is approximately 10 kHz, and the fine sampling resolution is 16-bit.

45) (canceled)

46) (canceled)

47) The system according to claim 2, wherein the implantable measurement device is configured to be operable in a receiving mode and a transmitting mode according to a transmission request;
wherein, optionally, when in the receiving mode, the implantable transceiver is configured to receive the transmission request from the external monitoring device;
wherein, optionally, when in the transmitting mode, the implantable transceiver is configured to transmit the event data to the external monitoring device.

48) (canceled)

49) (canceled)

50) The system according to claim 18, wherein the system further includes at least one of:
a) a plurality of implantable measurement devices;
b) one or more processing systems configured to:
i) at least partially analyse the subject data; and
ii) generate activity data indicative of results of the analysis;
c) a client device configured to interface with a user.

51) (canceled)

52) The system according to claim 50, wherein the one or more processing systems at least partially analyse the subject data using machine learning.

53) (canceled)

54) The system according to claim 50, wherein the client device at least one of:
a) includes:
i) a graphical user interface;
ii) a client device transceiver for receiving the subject data from the external monitoring device; and
iii) a client device display for displaying an activity indicator indicative of brain activity based on the subject data;
b) is one of a tablet, a smartphone, a smart watch and a computer; and
c) is configured to:
i) transmit subject data to one or more processing systems;
ii) receive activity data from the one or more processing systems; and,
iii) display an activity indicator based on the activity data.

55) (canceled)

56) (canceled)

57) (canceled)

58) (canceled)

59) (canceled)

60) (canceled)

61) (canceled)

62) (canceled)

63) (canceled)

64) (canceled)

65) (canceled)

66) (canceled)

67) (canceled)

68) (canceled)

69) (canceled)

70) (canceled)

71) (canceled)

72) (canceled)

73) (canceled)

74) (canceled)

75) (canceled)

76) (canceled)

77) (canceled)

78) (canceled)

79) (canceled)

80) (canceled)

81) (canceled)

82) (canceled)

83) (canceled)

84) (canceled)

85) (canceled)

86) (canceled)

87) (canceled)

88) (canceled)

89) (canceled)

90) (canceled)

91) (canceled)

92) (canceled)

93) (canceled)

94) (canceled)

95) (canceled)

96) (canceled)

97) (canceled)

98) (canceled)

99) (canceled)

100) (canceled)
